(19) United States
(12) Patent Application Publication
(10) Pub. No.: US 2005/0137409 A1
(43) Pub. Date: Jun. 23, 2005

(54) Processes for preparing benzoquinones and hydroquinones

(76) Inventors: Sunil Ashtekar, Bangalore (IN); Pramod Kumbhar, Mumbai (IN); Jan-Pleun Lens, Breda (NL); Rathinam Jothi Mahalingam, Bangalore (IN); Nadkarni Pradeep, Bangalore (IN); Jegadeesh Thampi, Bangalore (IN)

Correspondence Address: CANTOR COLBURN LLP, 55 GRIFFIN RD SOUTH, BLOOMFIELD, CT, 06002, US

(21) Appl. No.: 11/008,029

(22) Filed: Dec. 9, 2004

Related U.S. Application Data
(63) Continuation-in-part of application No. 10/757,921, filed on Jan. 14, 2004.
(60) Provisional application No. 60/530,562, filed on Dec. 18, 2003.

(51) Int. Cl. C07C 50/04; C07C 45/27

(52) U.S. Cl. 552/293; 568/772

(57) ABSTRACT

A process for the preparation of benzoquinones and hydroquinones includes oxidizing an aromatic hydroxy compound with oxygen or an oxygen containing gas, a copper containing catalyst, and optionally a promoter to form the benzoquinone. A reduction reaction is employed to form the hydroquinone.

### CROSS-REFERENCE TO RELATED APPLICATIONS

This application relates to and is a Continuation-In-Part of U.S. patent application Ser. No. 10/757,921, entitled “PROCESSES FOR PREPARING BENZOQUINONES AND HYDROQUINONES”, filed on Jan. 14, 2004, which claims priority to U.S. Provisional Application No. 60/530,562, filed on Dec. 18, 2003.

### BACKGROUND

This disclosure generally relates to a process for preparing hydroquinone compounds from aromatic hydroxy compounds.

Hydroquinone compounds find applications in a wide range of industries including, among others, the polymer industry, the dye industry, the photographic industry and in medical applications. They are also known for fabricating polycarbonates for use in liquid crystal displays.

Prior methods for the preparation of hydroquinone compounds generally include oxidation of aromatic hydroxy compound to the corresponding benzoquinone compound followed by reduction of the benzoquinone to give the corresponding hydroquinone compound. The oxidation of aromatic hydroxy compounds to benzoquinones has been widely studied and some of the earlier methods for oxidation typically include oxidation of aromatic hydroxy compounds with an inorganic oxidizing agent like potassium permanganate, manganese dioxide and lead oxide or with oxygen in the presence of a catalyst. However, the disadvantage associated with this method is the need for stoichiometric amounts of expensive oxidizing agents and the necessity to treat or regenerate the metals in a reduced valency state.

Other techniques employed in the preparation of substituted and unsubstituted benzoquinones include the use of catalyst systems containing copper. Aqueous solutions having cupric chloride and lithium chloride in 30-50% concentration (molar ratio phenol:cupric chloride dihydrate:lithium chloride:1:1:4) have been used to effect the oxidation of 2,3,6-trimethyl phenol to 2,3,5-trimethylbenzoquinone. However, use of such high quantities of catalyst leads to formation of undesired chloro-substituted compounds.

Copper containing catalysts in the presence of promoters like thiocyanate, cyanate, cyanide ions, or halogens in water and water miscible solvents have also been disclosed for the oxidation of phenols and alkyl substituted phenols to their corresponding benzoquinones. However, oxygen pressures employed are relatively high and the promoters are known toxic agents.

Use of high concentrations of about 20 to 80% of copper halogen complex Mₗ[Cu(II)ₘXₙ] combined with either an alkali metal halide or with an alkali metal halide and a cupric hydroxide and/or cuprous chloride in a medium of water and an aliphatic alcohol containing about 5 to about 10 carbon atoms to prepare 2,3,5-trimethylbenzoquinone from 2,3,6-trimethyl phenol are also disclosed. However, the molar ratio of copper halogen complex to phenol substrate is generally about 0.1-5:1, which leads to substantial formation of chloro compounds. One-pot oxidation processes to obtain various hydroquinones and substituted hydroquinones from phenol or substituted phenol are also disclosed, in that a divalent copper catalyst promoted with an alkali metal hydroxide or a monovalent copper catalyst promoted with water is used in the presence of oxygen, followed by hydrogenation with hydrogen gas at an elevated pressure and temperature in the same system after flushing out the oxygen gas. The quantity of oxygen and hydrogen used here is relatively high.

Accordingly, there is a need in the art for a commercial and cost effective process for manufacturing hydroquinones with high conversion and high selectivity.

### BRIEF SUMMARY

Disclosed herein is a process for preparing benzoquinone compounds, the process comprising oxidizing an aromatic hydroxy compound in a solvent with an oxygen gas or an oxygen-containing gas mixture in the presence of a catalytic amount of a copper containing catalyst and a promoter to form the benzoquinone compound, wherein said copper containing catalyst comprises a mixture of a halide salt and a copper salt, or a double salt of the halide salt and the copper salt, and wherein the catalytic amount of the copper containing catalyst less than or equal to 0.1 mole per mole of the aromatic hydroxy compound. The process may further comprise reducing the so-formed benzoquinone compounds to the corresponding hydroquinone compounds.

In another embodiment, a process for preparing 2-methylhydroquinone comprises oxidizing ortho-cresol with oxygen gas in a solvent selected from the group consisting of methylisobutylketone and methylethylketone in the presence of lithium trichlorocuprate dihydrate catalyst, and N-methylpyrrolidone promoter at a pH of about 1 to about 5 to form 2-methylbenzoquinone, wherein the lithium trichlorocuprate dihydrate catalyst is less than or equal to 0.1 mole per mole of the ortho-cresol; subsequently reducing the 2-methylbenzoquinone; and finally isolating the 2-methylhydroquinone.

In another embodiment a process for preparing hydroquinone compounds comprises oxidizing an aromatic hydroxy compound in a solvent with an oxygen gas or an oxygen-containing gas mixture in the presence of a catalytic amount of a copper containing catalyst and optionally a promoter to form the benzoquinone compound, wherein said copper containing catalyst comprises a mixture of a halide salt and a copper salt, or a double salt of the halide salt and the copper salt, and wherein the catalytic amount of the copper containing catalyst is less than or equal to 0.1 mole per mole of the aromatic hydroxy compound.

In yet another embodiment, a process for preparing 2-methylhydroquinone comprises oxidizing ortho-cresol with oxygen gas in an alcohol solvent comprising methanol in the presence of a mixture of cupric chloride dihydrate and sodium chloride and/or lithium chloride wherein the cupric chloride dihydrate catalyst is less than or equal to 0.1 mole per mole of the ortho-cresol; the sodium chloride is about 0.25 moles per mole of the ortho-cresol and the lithium chloride is about 0.25 moles per mole of the ortho-cresol.

In yet another embodiment, a process for preparing a benzoquinone compound comprises oxidizing an aromatic hydroxy compound in a solvent with an oxygen gas or an oxygen-containing gas mixture in the presence of a copper containing catalyst and optionally a promoter to form the benzoquinone compound, wherein said promoter comprises an aliphatic nitrile, an aromatic nitrile or an organic amide.

In yet another embodiment a process for preparing 2-methylbenzoquinone comprises oxidizing ortho-cresol with oxygen gas in an alcohol solvent comprising methanol in presence of cupric bromide and sodium bromide and optionally a promoter to form the benzoquinone compound, wherein the cupric bromide is less than or equal to 0.1 mole per mole of the ortho-cresol and the sodium bromide is about 0.5 moles per mole of the ortho-cresol.

The present disclosure may be understood more readily by reference to the following detailed description of the various features of the disclosure and the examples included therein.

### DETAILED DESCRIPTION

Disclosed herein is a process for preparing benzoquinone compounds that is cost effective with high conversion and high selectivity. The benzoquinone compounds are prepared from aromatic hydroxy compounds preferably of the formula: 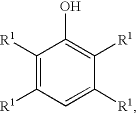
wherein R¹ is independently selected from the group consisting of a hydrogen and a hydrocarbyl group, wherein the hydrocarbyl group is selected from the group consisting of an alkyl group containing 1 to about 18 carbon atoms, an aryl group containing about 6 to about 20 carbon atoms, an aralkyl group containing about 6 to about 12 carbon atoms and an alkylaryl group containing about 7 to about 16 carbon atoms. The benzoquinone compounds are optionally reduced to provide the corresponding hydroquinone compounds. The hydroquinone compounds have a variety of applications, including use as monomer in the preparation of polycarbonates, as intermediate in the preparation of vitamins and dyestuffs, and in the photographic industry.

The process for the preparation of the benzoquinone compounds generally comprises oxidizing an aromatic hydroxy compound with oxygen gas or an oxygen-containing gas mixture in a solvent in the presence of a catalytic amount of a copper containing catalyst and optionally a promoter. The pressure and temperature during the oxidation is effective to form a benzoquinone compound. The so-formed benzoquinone compound could then be optionally reduced with a reducing agent to provide the corresponding hydroquinone compound. The hydroquinone compound may then be isolated using an anti-solvent.

The singular forms “a”, “an” and “the” include plural referents unless the context clearly dictates otherwise. “Optional” or “optionally” means that the subsequently described event or circumstance may or may not occur, and that the description includes instances where the event occurs and instances where it does not.

Unless otherwise specified, the term “alkyl” as used herein is intended to designate straight chain alkyls and branched chain alkyl groups. Illustrative non-limiting examples of suitable straight chain and branched chain alkyl groups include methyl ethyl, n-propyl, isopropyl, n-butyl, sec-butyl, tertiary-butyl, pentyl, neopentyl, hexyl, heptyl, octyl, nonyl, decyl, undecyl and dodecyl. Suitable aralkyl groups include, but are not intended to be limited to, benzyl, phenylbutyl, phenylpropyl, and phenylethyl. In various other embodiments, the term “aryl” or “aromatic” groups are intended to designate monocyclic or polycyclic moieties containing about 6 to about 20 ring carbon atoms. Some illustrative non-limiting examples of these aromatic groups include phenyl, biphenyl, and naphthyl.

The aromatic hydroxy compound is preferably oxidized directly to the corresponding benzoquinone in a solvent in the presence of a copper containing catalyst and optionally a promoter. The oxidation can be carried out using oxygen gas or an oxygen-containing gas mixture comprising nitrogen, ambient air, helium or argon. More specifically, the oxidation can be carried out using oxygen gas. The copper containing catalyst comprises a mixture of a halide salt and a copper salt, or a double salt of a halide salt and a copper salt. The promoter typically comprises an aliphatic nitrile, an aromatic nitrile or an organic amide. Alternatively, a copper salt alone can be used as a catalyst in combination with the organic amide promoter. During oxidation, especially in the presence of an organic amide as the promoter, a pH of about 1 to about 5 may be maintained. Additional acid may be added to maintain the pH. The so-formed benzoquinone may be subsequently reduced with a reducing agent to give the hydroquinone compound.

Specific examples of suitable aromatic hydroxy compounds include, but are not intended to be limited to, 2,6-dimethylphenol, 2,3,6-trimethylphenol, 2,6-di-tert-butylphenol, 2-tert-butylphenol, alpha-naphthol, meta-cresol, ortho-cresol, ortho-phenylphenol, ortho-chlorophenol, ortho-benzylphenol, 2,6-dichlorophenol, ortho-vinylphenol, and mixtures of the foregoing aromatic hydroxy compounds. In one particular embodiment, the aromatic hydroxy compound is ortho-cresol.

Specific examples of the halide salt include halide salts of the formula, M-X, wherein M comprises an alkali metal, an ammonium ion, or an organoammonium ion, and X is selected from the group consisting of chloride, bromide, and iodide. The organoammonium ion is specifically of the formula, R²—[NH₃]⁺, wherein R² is a monovalent hydrocarbyl group containing 1 to about 6 carbon atoms, and wherein the hydrocarbyl group includes, but is not limited, to isopropyl, n-butyl, tertiary butyl, and isopentyl groups.

Suitable halide salts include, but are not intended to be limited to, sodium chloride, lithium chloride, potassium chloride, cesium chloride, sodium bromide, ammonium bromide, potassium bromide, cesium bromide, sodium iodide, lithium iodide, potassium iodide, cesium iodide, isopropyl ammonium bromide, and mixtures of the foregoing halide salts. In one particular embodiment, the halide salt comprises sodium chloride, lithium chloride or mixture of sodium chloride and lithium chloride.

Specific examples of copper salts include, a cuprous salt, a cupric salt, or mixtures of the foregoing copper salts. More specifically the copper salts include, but are not limited to, cuprous chloride, cuprous bromide, cuprous iodide, cupric chloride, cupric bromide, cupric iodide, cuprous acetate, and cupric acetate. More particularly the copper salt is cupric chloride. Both anhydrous and hydrated forms of the copper salts may be used as the catalyst, like for example cupric chloride dihydrate or cupric chloride anhydrous.

Specific examples of a double salt of the halide salt and the copper salt include a compound of the formula, M[CuX₃], or a compound of the formula, M₂[CuX₄]; wherein M is selected from the group consisting of an alkali metal, an ammonium ion, and an organoammonium ion; and X is selected from the group consisting of chloride, bromide, and iodide. Alkali metals include, but are not limited to sodium, potassium, lithium, and cesium. The organoammonium ion is of the formula, R²— [NH₃]⁺; wherein R² is a monovalent hydrocarbyl group containing 1 to about 6 carbon atoms; wherein the hydrocarbyl group is selected from a group including isopropyl, isobutyl, butyl, tertiary butyl and isopentyl groups.

More specifically the double salts include, but are not intended to be limited to, lithium trichlorocuprate dihydrate, ammonium trichlorocuprate dihydrate, diammonium tetrachlorocuprate dihydrate, dipotassium tetrachlorocuprate dihydrate, cesium trichlorocuprate dihydrate, dicesium tetrachlorocuprate dihydrate, dilithium tetrabromocuprate hexahydrate, potassium tribromocuprate, diammonium tetrabromocuprate dihydrate, and cesium tribromocuprate. In one particular embodiment, the double salt is lithium trichlorocuprate dihydrate.

The double salts can be prepared by known procedures, for example by following the methods described in Mellor's Comprehensive Treatment on Inorganic and Theoretical Chemistry, 1963, Vol. III, pages 182-201 (Longman), the pages of which are incorporated herein by reference in their entirety.

The amount of the copper containing catalyst employed, when a double salt is used, may preferably comprise less than or equal to 0.1 moles per mole of aromatic compound, with 0.01 moles to 0.1 mole per mole of aromatic hydroxy compound more preferred, and with about 0.025 moles to about 0.075 moles per mole of aromatic hydroxy compound even more preferred. Likewise, when a combination of the halide salt and the copper salt is used, the ratio of the catalyst to the aromatic hydroxy compound may preferably comprise less than 0.1 moles per mole of aromatic hydroxy compound, with 0.01 moles to 0.1 mole per mole of aromatic hydroxy compound more preferred, and with about 0.025 moles to about 0.075 moles per mole of aromatic hydroxy compound even more preferred and the ratio of the halide salt to the aromatic hydroxy compound is less than or equal to 0.50 moles to 0.40 per mole of aromatic hydroxy compound, with about 0.35 to about 0.35 moles per mole of aromatic hydroxy compound more preferred, and with about 0.20 moles to about 0.25 moles of aromatic hydroxy compound even more preferred.

Specific examples of promoters include aliphatic nitriles containing 2 to 6 carbon atoms, aromatic nitriles containing 7 to 13 carbon atoms and organic amides. More specifically, the aliphatic nitriles include, but are not intended to be limited to acetonitrile and propiononitrile. More specifically the aromatic nitriles include but are not intended to be limited to benzylnitrile and benzonitrile. More specifically, the organic amides include, but are not intended to be limited to, N-methyl-2-pyrrolidone, N,N-dimethylformamide, N,N-diphenylformamide, N-cyclohexyl-N-methylformamide, N-methyl-acetamide and N-phenyl-N-methylformamide. The weight ratio of the promoter to the aromatic hydroxy compound is about 0.05 to about 0.7. In one particular embodiment, the weight ratio of promoter to aromatic hydroxy compound is about 0.25 to about 0.5.

Advantageously, the use of the promoter reduces the amount of catalyst employed in the oxidation of aromatic hydroxy compounds to corresponding benzoquinones, thereby leading to a significant reduction in the formation of undesired chloro compounds compared to prior art processes. Advantageously, the lower amounts of catalyst result in increased conversions and higher selectivities.

The oxidation of the aromatic hydroxy compound with the oxidizing agent can be carried out either in a water-miscible solvent or a partially water-miscible solvent. The water-miscible solvent is preferably selected from the group consisting of an alcohol containing 1 to about 8 carbon atoms, a sulfoxide containing about 2 to about 4 carbon atoms, and an ether containing about 4 to about 12 carbon atoms. Exemplary water-miscible solvents include, but are not limited to methanol, isopropyl alcohol, octanol, dimethylsulfoxide and monoethyleneglycol dimethyl ether (monoglyme). The partially water-miscible solvent comprises an organic ketone containing about 4 to about 10 carbon atoms. Exemplary partially water-miscible solvents include, but are not limited to, methyl isobutyl ketone and methyl ethyl ketone.

Typically when the oxidation of the aromatic hydroxy compound is carried out in presence of an organic amide as a promoter with the oxidizing agent, a pH of 1 to about 5 is maintained, and more particularly, a pH of about 2 can be maintained. The desired pH is maintained by optionally adding an acid. The acids that are optionally used to maintain the pH include, but are not limited to, a water-miscible organic acid, a water-miscible inorganic acid, or combinations of the foregoing acids. The organic acid is preferably an aliphatic carboxylic acid. Suitable aliphatic organic acids include, but are not limited to acetic acid, formic acid, oxalic acid, propionic acid and combinations of the foregoing acids. The inorganic acid is preferably a protic acid selected from the group of hydrochloric acid, sulfuric acid and combinations of the foregoing acids. Since acid is used optionally, the amount of acid employed in the reaction is sufficient enough for adjusting and maintaining the pH from 1 to about 5, more particularly at a pH of about 2.

The oxidation of the aromatic hydroxy compound with the oxidizing agent may be specifically carried out at a temperature of about 20° C. to about 75° C., and more particularly, at a temperature of about 30° C. to about 60° C. The pressure during the reaction is at about 34.5 Newtons per square centimeter (N/cm²) to about 345 N/cm², and more particularly, at a pressure of about 51.7 N/cm² to about 103 N/cm² and even more particularly, at a pressure of about 68.9 N/cm² to about 89.6 N/cm². The time required for the oxidation may vary from about 5 hours to about 24 hours, and more particularly, from about 5 hours to about 10 hours.

The oxidation of the aromatic hydroxy compound in presence of the catalyst and optionally a promoter takes place in a pressurized reactor vessel. Laboratory scale experiments may use, for example, a Parr pressurized reactor vessel commercially available from the Parr Instrument Company. The workup of the reaction mixture differs depending on whether the solvent used is partially water-miscible or water-miscible. When partially water-miscible solvent is used, the reaction mixture after oxidation step is subjected to a water wash treatment to remove the halide and copper salt or the double salt. Optionally, the organic layer including the partially water-miscible solvent containing the benzoquinone is used for the reduction step. When water-miscible solvent is used, the reaction mixture after oxidation is diluted with water and then extracted in a solvent such as toluene or xylene, the solvent is evaporated out to obtain a residue. Optionally, the residue is taken in a solvent suitable for reduction. The solvents for reduction can be selected from alcohols containing 1 to about 4 carbon atoms, acetic acid, and water. Suitable alcohols include, but are not intended to be limited to, methanol, ethanol, isopropyl alcohol, ethylene glycol, and mixtures of the foregoing alcohols.

Suitable reducing agents comprise hydrogen gas or hydrogen gas containing mixtures in presence of a reduction catalyst or the reducing agent. Suitable reducing agents are selected from the group consisting of sodium borohydride, sodium dithionate, lithium aluminum hydride, sodium hydrosulfite, iron powder, zinc powder and sodium bisulfite. Suitable reduction catalysts are selected from the group including Raney nickel, palladium-carbon, palladium supported on alumina, palladium supported on silica, platinum supported on charcoal, platinum supported on alumina, platinum supported on silica, tin, iron-hydrochloric acid, and zinc-acetic acid. After completion of the reduction, the solvent is distilled off and an anti-solvent is added to the residue to precipitate the hydroquinone compound.

The anti-solvent employed for isolation after the reduction step is selected from the group including, but not limited to, toluene, xylenes, chlorobenzenes, and heptane. In one particular embodiment, the anti-solvent is toluene.

As previously discussed, the hydroquinone compounds find various end use applications in the polymer, dyestuff, pharmaceutical, photographic industries, and in medical applications. For example, polycarbonates, particularly those containing methyl hydroquinone units, are known to exhibit liquid crystalline properties. Suitable methods for preparation of these polycarbonates include melt-transesterification reaction of diphenylcarbonate and mixtures of methyl hydroquinone and bisphenol; and melt polymerization methods in presence of quaternary phosphonium salts, sodium hydroxide or tetraalkylammonium salts as catalyst systems. The hydroquinone compounds could also be used to prepare polyesters when coupled with other monomers by melt polymerization techniques as is known in the art.

The disclosure is explained in more detail with reference to the following non-limiting examples, which are only illustrative, but not limitative.

### EXAMPLES

In the following examples and comparative examples, a high performance liquid chromatography (HPLC) method was used to quantify the conversion of an aromatic hydroxy compound to a benzoquinone compound. The HPLC was initially calibrated using standard samples of aromatic hydroxy compound and corresponding benzoquinone and hydroquinone commercially available from Aldrich Chemcial Company. The standard samples were diluted with an internal standard solution of N-methyl benzamide in acetonitrile and a sample injected into a C-18 reverse phase column. Each reaction mixture was then diluted with an internal standard solution of N-methyl benzamide in acetonitrile and a sample of which was injected into a C-18 reverse phase column. Samples at specific time intervals were analyzed and compared to the HPLC chromatogram of the standard sample to determine the conversion of aromatic hydroxy compound and selectivity towards corresponding benzoquinone after oxidation and hydroquinone after reduction. The pH is measured using a glass membrane electrode that is calibrated using 2 point calibration with standard buffers. The accuracy of the pH meter is +0.01.

### Example 1

In this example, a lithium trichlorocuprate dihydrate catalyst stock solution was prepared. A mixture of cupric chloride (40.02 gm) and lithium chloride (9.97 gm) was placed in a 100 milliliter (ml) volumetric flask and the volume made up to 100 ml with water.

### Example 2

In this example, 2-methyl hydroquinone was prepared. A mixture of ortho-cresol (127 grams), lithium trichorocuprate dihydrate solution (22.3 milliliters) as prepared in Example 1, N-methylpyrrolidone (41.81 grams), acetic acid (41.81 grams), and methyl isobutyl ketone (842.64 grams) was charged to a 3.7 liter Parr pressure vessel. The pressure vessel was closed and pressurized with oxygen to 35 N/cm² and depressurized to atmospheric pressure. This was repeated thrice. The reactor was heated to 50° C. and pressurized with oxygen to a pressure of 70 N/cm². This pressure was maintained throughout the experiment by replenishment with oxygen as needed. The reaction was monitored by high-performance liquid chromatography (HPLC) for the conversion of ortho-cresol and the selectivity of conversion to methyl benzoquinone. The reactor was cooled to room temperature (25° C.) and the reaction mixture was washed three times with water. The water quantity used each time for the washing was equal to about 25 percent of the total weight of the reaction mixture.

To effect reduction to the corresponding hydroquinone, a mixture of the above washed reaction mixture (200 grams (g), and Raney Nickel water slurry (3.6 g) (approx 50% wt/wt nickel to water) was charged to a 600 milliliter Parr pressure vessel and the pressure vessel was closed and pressurized with nitrogen to 35 N/cm² and depressurized to atmospheric pressure. This was repeated thrice. The reactor was heated to 80° C. and pressurized with hydrogen to a pressure of 63 N/cm². This pressure was maintained throughout the experiment by replenishment with hydrogen. The reaction was monitored by HPLC for the conversion of methyl benzoquinone and the selectivity of conversion to methyl hydroquinone. The reactor was cooled and the reaction mixture was distilled to remove the methyl isobutyl ketone solvent. The residue was used for the isolation and purification of methyl hydroquinone. The weight of the residue obtained was about 34.59 g.

To the above residue was added toluene (100 ml) and the resultant mixture was stirred for 12 hours at room temperature (25° C.). The precipitated methyl hydroquinone was filtered and the mother liquor cooled to about 10° C. to recover a second crop of the methyl hydroquinone. Total crude product obtained was 8 grams (g), which was subsequently washed with 4 volumes of toluene and dried.

### Example 3

In this example, 2-methyl hydroquinone was prepared by following the same procedure as mentioned in Example 2 except that ortho-cresol (61.97 grams), lithium trichoro cuprate dihydrate solution (11.99 g), N,N-dimethylformamide (20.88 g), acetic acid (11.27 g) and methyl isobutyl ketone (419.12 g) were used. Reduction of the methyl benzoquinone reaction mixture was carried out in the same manner as described in Example 2, except in this case the entire reaction mixture was taken for reduction. After about 5.5 hrs, the product was isolated in the same manner as in Example 2. The yield of pure product obtained was 16.46 grams.

### Examples 4-7

In these examples, 2-methyl benzoquinone was prepared using the components set forth in Table 1 and the reaction parameters set forth in Table 2.

The general procedure followed in these reactions includes charging a mixture of the aromatic hydroxy compound, catalyst, promoter, acid, and solvent in a 450 milliliter Parr pressure vessel under a continuous flow of oxygen at 300 milliliter per hour. The reactor is heated and pressurized with oxygen to a pressure of 70 N/cm². An acidic pH is maintained by addition of acetic acid. These reactions were carried out in methyl isobutyl ketone to study the effect of temperature on the conversion and selectivity in the oxidation of ortho-cresol to 2-methyl benzoquinone.

TABLE 1

| | | | | | Catalyst |
|---|---|---|---|---|---|
| | | | | | Lithium |
| | | | | | trichloro |
| | Aromatic | Solvent | | | cuprate |
| | hydroxy | Methyl | Promoter | | dihydrate |
| | phenol | isobutyl | N-methyl | Acid | aqueous |
| | Ortho- | ketone | pyrrolidone | acetic acid | solution |
| Ex. No. | cresol (g) | (g) | (g) | (g) | (g) |
| 4 | 16.95 | 79 | 4.040 | 4.080 | 2.950 |
| 5 | 12.78 | 82.6¹ | 4.240 | 4.270 | 2.215 |
| 6 | 14.38 | 81.4 | 4.080 | 4.170 | 2.840 |
| 7 | 14.9046 | 81.4 | 5.126 | 5.261 | 4.0090 |

¹methyl ethyl ketone

TABLE 2

| Example | Temperature | | Time |
|---|---|---|---|
| No. | (° C.) | pH | (hours) |
| 4 | 42 | 2.1 | 8 |
| 5 | 50 | 2.1 | 12 |
| 6 | 55 | 1.9 | 8 |
| 7 | 60 | 1.5 | 5 |

### Examples 8-9

In these examples, 2-methyl benzoquinone was prepared using the components set forth in Table 3 and the reaction parameters set forth in Table 4 following the general procedure followed in Examples 4-7.

TABLE 3

| | | | | | Catalyst |
|---|---|---|---|---|---|
| | | | | | Lithium |
| | | | | | trichloro |
| | Aromatic | Solvent | | | cuprate |
| | hydroxy | Methyl | Promoter | | dihydrate |
| | phenol | isobutyl | N-methyl | Acid | aqueous |
| | Ortho- | ketone | pyrrolidone | acetic acid | solution |
| Ex. No. | cresol (g) | (g) | (g) | (g) | (g) |
| 8 | 14.3844 | 80.9 | 4.080 | — | 0.83 |
| 9 | 12.86 | 83.2 | 4.160 | 4.170 | 4.44 |

TABLE 4

| Example | Temperature | | Time |
|---|---|---|---|
| No. | (° C.) | pH | (hours) |
| 8 | 60 | 2.1 | 5 |
| 9 | 50 | 3.1 | 2 |

### Examples 10-11

In these examples, benzoquinones were prepared using the components set forth in Table 5 and the reaction parameters set forth in Table 6 following the general procedure followed in Examples 4-7. The results, as shown in Table 6, illustrate the effect of reaction conditions on phenol and 2,6-dimethylphenol.

TABLE 5

| | | | | | Catalyst |
|---|---|---|---|---|---|
| | | | | | Lithium |
| | | | | | trichloro |
| | Aromatic | Solvent | | | cuprate |
| | hydroxy | methyl | Promoter | | dihydrate |
| | phenol | isobutyl | N-methyl | Acid | aqueous |
| | Ortho- | ketone | pyrrolidone | acetic acid | solution |
| Ex. No. | cresol (g) | (g) | (g) | (g) | (g) |
| 10 | 13.040¹ | 79.4 | 4.880 | 4.990 | 2.00 |
| 11 | 17.27² | 80.2 | 4.890 | 5.020 | 2.00 |

¹phenol
²2,6-dimethyl xylenol

TABLE 6

| Example | Temperature | | Time |
|---|---|---|---|
| No. | (° C.) | pH | (hours) |
| 10 | 60 | 1.8 | 6 |
| 11 | 60 | 2.1 | 6 |

### Examples 12-20

In these examples, 2-methyl benzoquinone was prepared using the components set forth in Table 7 and the reaction parameters set forth in Table 8 following the general procedure followed in Examples 4-7. These examples illustrate the effect of water-miscible solvent, different catalyst, different promoter different acid, higher pressure and no acid on the formation of 2-methyl benzoquinone from ortho-cresol.

TABLE 7

| | | | | | Catalyst |
|---|---|---|---|---|---|
| | | | | | Lithium |
| | | | | | trichloro |
| | Aromatic | Solvent | | | cuprate |
| | hydroxy | methyl | Promoter | | dihydrate |
| | compound: 1 | isobutyl | N-methyl | Acid | aqueous |
| Ex. | ortho-cresol | ketone | pyrrolidone | acetic acid | solution |
| No. | (g) | (g) | (g) | (g) | (g) |
| 12 | 12.820 | 83.5³ | 4.2 | 4.230 | 2.22 |
| 13 | 24.89 | 168.9 | 4.890 | 5.020 | 3.24⁴ |
| 14 | 12.22 | 83.2 | 4.310⁵ | 4.270 | 2.115 |
| 15 | 25.64 | 165.8 | 8.32 | 8.31⁶ | 2.78 |
| 16 | 10.4861 | 98⁷ | 5.144 | 5.209 | 1.609 |
| 17 | 12.7 | 83.4 | 4.280 | 4.140 | 2.3180⁸ |
| 18 | 15.1 | 82 | 5.211 | 5.240 | 1.496⁹ |
| 19¹⁰ | 14.495 | 82.6 | 5.144 | 5.206 | 5.079 |
| 20¹¹ | 15.04 | 85.2 | 5.240 | — | 2.01 |

³monoglyme
⁴isorpropylammonium trichloro cuprate dihydrate
⁵N,N-dimethylformamide
⁶propionic acid
⁷methyl ethyl ketone
⁸cupric chloride dihydrate solution
⁹The amount of catalyst corresponds to cupric chloride dihydrate (1.1985 grams) and lithium chloride (0.2977 grams)
¹⁰oxygen pressure 105 N/cm²
¹¹no acid used

TABLE 8

| Example | Temperature | | Time |
|---|---|---|---|
| No. | (° C.) | pH | (hours) |
| 12 | 55 | 3.1 | 10 |
| 13 | 55 | 3.7 | 5 |
| 14 | 50 | 2.1 | 6 |
| 15 | 55 | 3.5 | 9 |
| 16 | 60 | 2.1 | 5 |
| 17 | 50 | 3.0 | 6 |
| 18 | 55 | 1.7 | 7 |
| 19 | 55 | 2.1 | 4 |
| 20 | 60 | 3.2 | 4 |

### Comparative Examples 1-2

In these examples, 2-methyl benzoquinone was prepared using the components set forth in Table 9 and the reaction parameters set forth in Table 10 following the general procedure of Examples 4-7. The conversions and selectivities are included in Table 10 below. The oxidation processes of comparative Examples 1 and 2 were without the promoter.

TABLE 9

| | | | | | Catalyst |
|---|---|---|---|---|---|
| | | | | | Lithium |
| | | | | | trichloro |
| | | Solvent | | | cuprate |
| | Substrate | Methyl | Promoter | Acid | dihydrate |
| | Ortho- | isobutyl | N-methyl | Acetic | aqueous |
| Comparative | cresol | ketone | pyrrolidone | acid | solution |
| Examples | (g) | (g) | (g) | (g) | (g) |
| 1 | 10.83 | 137¹ | — | — | 1.6343 |
| 2 | 20.36 | 100² | — | — | 1.06 |

¹methyl ethyl ketone
²isopropyl alcohol

TABLE 10

| Comparative | Temperature | | Time |
|---|---|---|---|
| Examples | (° C.) | pH | (hours) |
| 1 | 60 | 2.1 | 5 |
| 2 | 70 | 1.6 | 17 |

### Examples 21-31

In these examples, 2-methyl hydroquinone was prepared. A mixture of ortho-cresol, cupric chloride dihydrate, sodium chloride and/or lithium chloride, acetonitrile and methanol was charged to a 1 liter Parr pressure vessel. The pressure vessel was closed and pressurized with oxygen to 89.6 N/cm². The reactor was heated the pressure was maintained throughout the experiment by replenishment with oxygen as needed. The reaction was monitored by high-performance liquid chromatography (HPLC) for the conversion of ortho-cresol and the selectivity of conversion to methyl benzoquinone. The examples were carried out using the reaction contents and reaction parameters set forth in Table 11.

TABLE 11

| | | Cupric | | | | | | | ortho- | Methyl |
|---|---|---|---|---|---|---|---|---|---|---|
| | ortho- | chloride | Sodium | Lithium | Aceto- | | | | cresol | Benzoquinone |
| Ex. | cresol | dihydrate | Chloride | Chloride | nitrile | Methanol | Temp. | Time | conversion | Selectivity |
| No. | (g) | (g) | (g) | (g) | (g) | (g) | (° C.) | (hours) | (%) | (%) |
| 21 | 40.12 | 6.39 | 5.41 | 0 | 7.6 | 337 | 40 | 5 | 73 | 66 |
| | | | | | | | | 7 | 92 | 65 |
| 22 | 40.04 | 6.31 | 5.41 | 0 | 7.6 | 337 | 30 | 6 | 73 | 68 |
| 23 | 40.06 | 12.6 | 10.8 | 0 | 7.71 | 320 | 40 | 2 | 64 | 58 |
| | | | | | | | | 5 | 94 | 58 |
| | | | | | | | | 6 | 100 | 58 |
| 24 | 40.3 | 9.45 | 8.115 | 0 | 7.6 | 350 | 30 | 5 | 65 | 63 |
| | | | | | | | | 6 | 76 | 62 |
| 25 | 40.09 | 6.3113 | 5.4193 | 0 | 7.63 | 337 | 30 | 7 | 68 | 68 |
| | | | | | | | | 8 | 75 | 68 |
| 26 | 40.87 | 6.31 | 5.41 | 0 | 51.68 | 300 | 30 | 5 | 72 | 69 |
| | | | | | | | | 7 | 87 | 68 |
| 27 | 40.3 | 6.3 | 0 | 3.92 | 7.67 | 337 | 30 | 10 | 48 | 80 |
| | | | | | | | | 14 | 63 | 77 |
| | | | | | | | | 15 | 68 | 73 |
| 28 | 40.52 | 6.3 | 0 | 3.92 | 7.64 | 337 | 40 | 7 | 54 | 73 |
| | | | | | | | | 10 | 83 | 69 |
| | | | | | | | | 12 | 92 | 68 |
| | | | | | | | | 14 | 96 | 67 |
| 29 | 40 | 6.3 | 0 | 3.92 | 15.43 | 330 | 30 | 7 | 54 | 77 |
| 30 | 40.02 | 6.3 | 2.7 | 1.96 | 7.73 | 337 | 30 | 13 | 67 | 72 |
| 31 | 40.01 | 6.3 | 2.71 | 1.97 | 7.74 | 339 | 40 | 5 | 59 | 70 |
| | | | | | | | | 7 | 75 | 68 |

Examples 21-31 indicate that the use of acetonitrile as the promoter in conjunction with cupric chloride dihydrate and sodium and/or lithium chloride provides ortho-cresol conversions of greater than 50 percent and a selectivity of conversion to methyl benzoquinone of greater than 55 percent.

### Examples 32-41

In these examples, 2-methyl benzoquinone was prepared using the components and reaction parameters set forth in Table 12. In these examples, the effect of catalyst type and catalyst loading on the oxidation of ortho-cresol was studied. The reaction contents and reaction parameters used are set forth in Table 12. The temperature was maintained at 40° C. The conversion of ortho-cresol and the selectivity of conversion to methyl benzoquinone at 5 hours were monitored using HPLC. The factors analyzed were catalyst (copper halide type) loading and alkali metal halide type. The catalyst loading varied from 2 to 5 mole % based on o-cresol and the two alkali metal halides studied were sodium bromide and sodium chloride.

TABLE 12

| | | Cupric | | | | | | Methyl |
|---|---|---|---|---|---|---|---|---|
| | Ortho- | chloride | Cupric | Sodium | Sodium | | Ortho-cresol | Benzoquinone |
| | cresol | dihydrate | Bromide | Chloride | Bromide | Methanol | conversion | Selectivity |
| Example | (g) | (g) | (g) | (g) | (g) | (g) | (%) | (%) |
| 32 | 40.12 | 3.16 | — | — | 9.53 | 350 | 43 | 74 |
| 33 | 40.01 | 3.16 | — | 5.41 | — | 350 | 44 | 64 |
| 34 | 40.26 | 1.27 | — | 5.41 | — | 352 | 10 | 100 |
| 35 | 40.04 | 2.21 | — | 5.41 | — | 350 | 15 | 100 |
| 36 | 40.18 | 1.26 | — | — | 9.53 | 352 | 15 | 100 |
| 37 | 40.02 | — | 2.9 | 5.41 | — | 352 | 23 | 77 |
| 38 | 40.04 | — | 2.9 | — | 9.53 | 350 | 26 | 73 |
| 39 | 40.24 | — | 1.65 | — | 9.54 | 351 | 9 | 86 |
| 40 | 40.13 | — | 1.65 | 5.41 | — | 352 | 1 | 100 |
| 41 | 40.15 | — | 4.14 | — | 9.53 | 355 | 45 | 57 |

Examples 32-41 demonstrate that the process generally provides high selectivity in the conversion of ortho-cresol to the corresponding aromatic hydroxy compound when bromide or chloride containing copper catalyst and alkali metal halide salts are employed. It should be noted that the experiments conducted as part of this study were not optimized in all cases. Thus, it is believed that much higher conversion of ortho-cresol than those shown in Table 12 are achievable for examples which show less than 50 percent conversion, by adjusting various reaction parameters which are known to those skilled in the art. Such optimization is well within the skill of those in the art.

### Examples 42-48

In these examples, the temperature and catalyst loading were varied to study the effect on the oxidation of ortho-cresol. Cupric chloride was used as the catalyst and the alkali metal halide used was sodium chloride. Pressure was maintained at 89.6 N/cm². The reaction contents and other reaction parameters used are set forth in Table 13.

TABLE 13

| | | Cupric | | | | | Methyl |
|---|---|---|---|---|---|---|---|
| | Ortho- | chloride | Sodium | | | Ortho-cresol | Benzoquinone |
| | cresol | dihydrate | bromide | Methanol | Temperature | conversion | Selectivity |
| Example | (g) | (g) | (g) | (g) | (° C.) | (%) | (%) |
| 42 | 40.14 | 6.31 | 5.41 | 350 | 30 | 66 | 65 |
| 43 | 40.02 | 3.16 | 5.42 | 350 | 30 | 27 | 71 |
| 44 | 40.06 | 4.74 | 5.41 | 350 | 40 | 68 | 57 |
| 45 | 40.06 | 4.74 | 5.41 | 351 | 40 | 67 | 59 |
| 46 | 40.09 | 4.74 | 5.42 | 350 | 40 | 79 | 49 |
| 47 | 40.02 | 3.15 | 5.41 | 353 | 50 | 48 | 68 |
| 48 | 40.06 | 6.31 | 5.41 | 352 | 50 | 100 | 55 |

Examples 42-48 indicate that varying the temperature of the oxidation reaction between 30° C. to 50° C. and the catalyst quantity from 5 to 10 mole percent based on the amount of ortho-cresol provided good conversions of ortho-cresol and good selectivities to methyl hydroquinone.

### Examples 49-51

In these examples, lithium trichlorocuprate dihydrate catalyst solution as prepared in Example 1 was employed as the catalyst using different solvent systems. The system was pressurized with oxygen gas at a pressure of about 345 N/cm². The reaction contents and reaction parameters are enclosed in Table 14 below.

TABLE 14

| | | Lithium | | | | | |
|---|---|---|---|---|---|---|---|
| | | trichloro | | NMP with | | | Methyl |
| | Ortho- | cuprate | | respect to | Temperature | ortho-cresol | Benzoquinone |
| | cresol | dihydrate | | solvent | in | conversion | Selectivity |
| Example | (g) | (wt %) | Solvent | (%) | (° C.) | (%) | (%) |
| 49 | 25.6 | 11 | MIBK | 5 | 55 | 73 | 56 |
| 50 | 40.6 | 10 | Methanol | 0 | 40 | 98 | 71 |
| 51 | 51 | 10 | tertiary-butanol | 0 | 40 | 80 | 82 |

While the disclosure has been described with reference to an exemplary embodiment, it will be understood by those skilled in the art that various changes may be made and equivalents may be substituted for elements thereof without departing from the scope of the disclosure. In addition, many modifications may be made to adapt a particular situation or material to the teachings of the disclosure without departing from the essential scope thereof. Therefore, it is intended that the disclosure not be limited to the particular embodiment disclosed as the best mode contemplated for carrying out this invention, but that the disclosure will include all embodiments falling within the scope of the appended claims.

1. A process for preparing a benzoquinone compound, said process comprising:
oxidizing an aromatic hydroxy compound in a solvent with an oxygen gas or an oxygen-containing gas mixture in the presence of a catalytic amount of a copper containing catalyst and a promoter to form the benzoquinone compound, wherein said copper containing catalyst comprises a mixture of a halide salt and a copper salt, or a double salt of the halide salt and the copper salt, and wherein the catalytic amount of the copper containing catalyst is less than or equal to 0.1 mole per mole of the aromatic hydroxy compound.

2. The process of claim 1, further comprising reducing said benzoquinone compound with a reducing agent to provide a hydroquinone compound.

3. The process of claim 1, wherein said aromatic hydroxy compound has a formula: 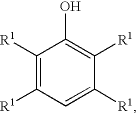
wherein R¹ is independently selected from the group consisting of a hydrogen and a hydrocarbyl group.

4. The process of claim 1, wherein said aromatic hydroxy compound is selected from the group consisting of phenol, 2,6-dimethyl phenol, 2,6-di-tertiary-butyl phenol, 2-tertiary-butyl phenol, alpha-naphthol, meta-cresol, ortho-cresol, ortho-phenylphenol, ortho-benzylphenol, 2,3,6-trimethyl phenol, ortho-vinylphenol, 2-isopropylphenol, 2,6-diisopropylphenol, 2,3,5,6-tetramethylphenol, 2,3,5-trimethylphenol, and mixtures of the foregoing aromatic hydroxy compounds.

5. The process of claim 1, wherein said aromatic hydroxy compound is ortho-cresol.

6. The process of claim 1, wherein said oxygen-containing gas mixture comprises nitrogen, ambient air, helium, or argon.

7. The process of claim 1, wherein said halide salt is of the formula, M-X;
wherein M comprises an alkali metal, an ammonium ion, or an organoammonium ion;
and X is selected from the group consisting of chloride, bromide, and iodide.

8. The process of claim 7, wherein said organoammonium ion is of the formula, R²— [NH₃]⁺, wherein R² is a monovalent hydrocarbyl group containing 1 to about 6 carbon atoms.

9. The process of claim 1, wherein said copper salt comprises a cuprous salt, a cupric salt, or mixtures of the foregoing copper salts.

10. The process of claim 1, wherein said copper salt is selected from the group consisting of cuprous chloride, cuprous bromide, cuprous iodide, cupric chloride, cupric bromide, cupric iodide, cuprous acetate, and cupric acetate.

11. The process of claim 1, wherein said double salt of said halide salt and said copper salt comprises a compound of the formula, M[CuX₃], or a compound of the formula, M₂[CuX₄]; wherein M is selected from the group consisting of an alkali metal, an ammonium ion, and an organoammonium ion; and X is selected from the group consisting of chloride, bromide, and iodide.

12. The process of claim 1, wherein said double salt of said halide salt and said copper salt is at least one selected from the group consisting of lithium trichlorocuprate dihydrate, ammonium trichlorocuprate dihydrate, diammonium tetrachlorocuprate dihydrate, dipotassium tetrachlorocuprate dihydrate, cesium trichlorocuprate dihydrate, dicesium tetrachlorocuprate dihydrate, dilithium tetrabromocuprate hexahydrate, potassium tribromocuprate, diammonium tetrabromocuprate dihydrate, and cesium tribromocuprate.

13. The process of claim 1, wherein said double salt of said halide salt and said copper salt is lithium trichlorocuprate dihydrate.

14. The process of claim 1, wherein oxidizing said aromatic hydroxy compound is carried out at a pH from 1 to about 5.

15. The process of claim 1, further comprising adjusting the pH during oxidation to maintain said pH at about 1 to about 5.

16. The process of claim 1, further comprising adding an acid and maintaining a pH at about 1 to about 5.

17. The process of claim 1, wherein said promoter comprises an aliphatic nitrile, an aromatic nitrile or an organic amide.

18. The process of claim 1, wherein said promoter is selected from the group consisting of N-methyl-2-pyrrolidone, N,N-dimethylformamide, N,N-diphenylformamide, N-methyl-acetamide, N-cyclohexyl-N-methylformamide, and N-phenyl-N-methylformamide, acetonitrile, propiononitrile, benzylnitrile and benzonitrile.

19. The process of claim 1, wherein oxidizing the aromatic hydroxy compound is carried out at a temperature of about 20° C. to about 75° C.

20. The process of claim 1, wherein oxidizing the aromatic hydroxy compound is carried out under a pressure of about 35 Newtons per square centimeter to about 345 Newtons per square centimeter.

21. The process of claim 1, wherein oxidizing the aromatic hydroxy compound is carried out in presence of a water-miscible solvent.

22. The process of claim 1, wherein oxidizing the aromatic hydroxy compound is carried out in presence of a partially water-miscible solvent.

23. The process of claim 22, wherein said partially water-miscible solvent comprises an organic ketone solvent containing about 4 to about 10 carbon atoms or combinations of said organic ketone solvents.

24. The process of claim 21, wherein said water-miscible solvent comprises an organic alcohol containing 1 to about 8 carbon atoms, an organic sulfoxide containing about 2 to about 4 carbon atoms, an organic ether containing about 4 to about 12 carbon atoms, or combinations of the foregoing solvents.

25. The process of claim 21, wherein said water-miscible solvent is selected from the group consisting of methanol, Isopropyl alcohol, octanol, dimethylsulfoxide, monoethylene glycol dimethyl ether, and combinations of the foregoing solvents.

26. The process of claim 22, wherein said partially water-miscible solvent is selected from the group consisting of methylisobutylketone, methylethylketone, and combinations of the foregoing solvents.

27. The process of claim 1, wherein said promoter and aromatic hydroxy compound are present in a weight ratio from about 0.05 to about 0.7.

28. The process of claim 2, wherein reducing said benzoquinone comprises:
contacting the benzoquinone with a reducing agent comprising hydrogen gas or hydrogen-containing gas mixtures in the presence of a reducing catalyst.

29. The process of claim 2, wherein the reducing agent is selected from the group consisting of sodium borohydride, sodium dithionate, lithium aluminum hydride, sodium hydro sulfite, iron powder, zinc powderand sodium bisulfite.

30. The process of claim 2, further comprising isolating the hydroquinone compound comprising:
distilling the solvent; and
precipitating said hydroquinone compound using an anti-solvent.

31. The process of claim 30, wherein said anti-solvent is selected from the group consisting of toluene, xylene, and heptane.

32. A polycarbonate comprising structural units derived from the hydroquinone compound prepared in accordance with claim 2.

33. A polycarbonate prepared using the hydroquinone compound prepared in accordance with claim 2.

34. A process for preparing 2-methylhydroquinone, said process comprising:
oxidizing ortho-cresol with oxygen gas in a solvent selected from the group consisting of methylisobutylketone and methylethylketone in the presence of lithium trichlorocuprate dihydrate catalyst, and N-methylpyrrolidone promoter at a pH of about 1 to about 5 to form 2-methyl benzoquinone, wherein the lithium trichlorocuprate dihydrate catalyst is less than or equal to 0.1 mole per mole of the ortho-cresol;
reducing the 2-methyl benzoquinone; and
isolating the 2-methylhydroquinone.

35. A polycarbonate comprising structural units derived from the 2-methylhydroquinone prepared in accordance with claim 35.

36. A process for preparing a benzoquinone compound, said process comprising:
oxidizing an aromatic hydroxy compound in a solvent with an oxygen gas or an oxygen-containing gas mixture in the presence of a catalytic amount of a copper containing catalyst and a promoter to form the benzoquinone compound, wherein said promoter comprises an aliphatic nitrile, an aromatic nitrile or an organic amide.

37. The process of claim 36, wherein the catalytic amount of the copper containing catalyst is less than or equal to 0.1 moles per mole of the aromatic hydroxy compound.

38. A process for preparing a benzoquinone compound, said process comprising:
oxidizing an aromatic hydroxy compound in a solvent with an oxygen gas or an oxygen-containing gas mixture in the presence of a catalytic amount of a copper containing catalyst and optionally a promoter to form the benzoquinone compound, wherein said copper containing catalyst comprises a mixture of a halide salt and a copper salt, or a double salt of the halide salt and the copper salt, and wherein the catalytic amount of the copper containing catalyst is less than or equal to 0.1 moles per mole of the aromatic hydroxy compound.

39. A process for preparing 2-methylhydroquinone, said process comprising:
oxidizing ortho-cresol with oxygen gas in an alcohol solvent comprising methanol in presence of cupric chloride dihydrate and sodium and/or lithium chloride and a promoter to form the benzoquinone compound, wherein the cupric chloride dihydrate catalyst is less than or equal to 0.1 moles per mole of the ortho-cresol, the sodium chloride is about 0.25 moles per mole of the ortho-cresol and/or the lithium chloride is about 0.25 moles per mole of the ortho-cresol.

40. A process for preparing 2-methylhydroquinone, said process comprising:
oxidizing ortho-cresol with oxygen gas in a solvent comprising methanol in the presence of cupric chloride dihydrate or cupric bromide and sodium chloride or sodium bromide and/or lithium chloride and optionally a promoter to form the benzoquinone compound, wherein the cupric bromide is less than or equal to 0.1 moles per mole of the ortho-cresol; the sodium chloride or the sodium bromide is about 0.25 moles per mole of the ortho-cresol, and the lithium chloride is about 0.25 moles per mole of the ortho-cresol.
